(19) United States
(12) Patent Application Publication
(10) Pub. No.: US 2014/0180030 A1
(43) Pub. Date: Jun. 26, 2014

(54) INTRAVASCULAR BLOOD PRESSURE AND VELOCITY WIRE

(71) Applicant: VOLCANO CORPORATION, San Diego, CA (US)

(72) Inventor: Dale Dorando, Shingle Springs, CA (US)

(73) Assignee: VOLCANO CORPORATION, San Diego, CA (US)

(21) Appl. No.: 14/132,382

(22) Filed: Dec. 18, 2013

Related U.S. Application Data
(60) Provisional application No. 61/740,056, filed on Dec. 20, 2012.

(51) Int. Cl. A61B 5/0215; A61B 5/026

(52) U.S. Cl. CPC A61B 5/0215; A61B 5/026
USPC 600/301; 600/486; 600/505

(57) ABSTRACT

The present invention generally relates to devices for determining pressure and flow in a vessel and methods for using such devices. The device can involve an elongate body configured for insertion into a vessel, a flow sensor positioned on the elongate body configured for detecting flow in the vessel, and a pressure sensor positioned on the elongate body configured for detecting pressure in the vessel, wherein the pressure sensor comprises an optical sensor.

### CROSS-REFERENCE TO RELATED APPLICATION

This application claims priority to, and the benefit of, U.S. Provisional Patent Application Ser. No. 61/740,056, filed Dec. 20, 2012, the contents of which are incorporated by reference.

### FIELD OF THE INVENTION

The present invention generally relates to medical devices for detecting both pressure and flow in a vessel and methods of using such devices.

### BACKGROUND

Cardiovascular disease frequently involves the buildup of deposits on the walls of blood vessels, particularly the arterial lumen of the coronary and other vasculature, resulting in a condition known as atherosclerosis. These deposits can have widely varying properties, with some deposits being relatively soft and others being fibrous and calcified as plaque. These deposits can restrict blood flow, leading to heart attack and stroke.

The assessment and treatment of cardiovascular disease often involves determining the relative pressure within the vessel and the speed at which blood moves through the vessel, also known as flow. These methods typically involve the use of a guidewire inserted to blood vessel to measure such parameters, which are subsequently used to determine other criteria, such as Fractional Flow Reserve (FFR). FFR is a measurement of the maximum myocardial flow in the presence of a stenosis (i.e., a narrowing of the blood vessel) divided by the normal maximum myocardial flow. This ratio is approximately equal to the mean hyperemic (i.e., dilated vessel) distal coronary pressure divided by the mean arterial pressure. An FFR below a certain value typically indicates that therapeutic intervention is required.

The assessment of pressure and flow typically involves the use of separate guidewires for pressure and flow measurement or may involve the use of a single guidewire that can measure both pressure and flow. While the use of a single guidewire with combined capabilities is certainly advantageous, certain aspects of these combination wires are still less than optimal. For example, certain conventional combination wires use resistive components for measuring pressure that are prone to temperature and moisture drift. This drift can significantly affect the accuracy of pressure readings. As a result, efforts to properly diagnose the extent of cardiovascular disease and subsequent identify appropriate treatment are hindered.

### SUMMARY

The present invention relates to combination intravascular devices, such as guidewires and catheters, for detection of both pressure and flow inside a vessel. In contrast to conventional combination guidewires, however, the provided devices use optical elements to assess pressure rather than resistive elements. The invention recognizes that optical sensors are less sensitive to temperature and moisture drift compared to resistance-based sensors, therefore, the use of optical sensors in the provided devices results in improved accuracy and greater performance. This in turn leads to increased confidence when diagnosing a stenosis and facilitates identification of the appropriate treatment.

Although devices in accordance with the invention may include any elongate body, such as a catheter, guidewires are particularly useful for practicing the invention. A physician, for example, may use the provided guidewire to determine the FFR of the vessel, and should the result warrants treatment, the physician may then slide a catheter over the guidewire to deliver a stent or balloon.

In certain aspects of the invention, the guidewire encompasses a hollow guidewire with the optical fiber placed inside. In certain aspects, the optical fiber may include a Fabry-Perot interferometer to determine pressure. In a typical Fabry-Perot interferometer or cavity, two partially reflective surfaces are aligned with each other such that many waves of light derived from the same incident wave can interfere. The resultant interference patterns may be used to analyze the spectral character of the incident beam. In further aspects of the invention, the optical fiber includes at least one fiber Bragg grating (FBG) for transmitting light in desired configurations along the optical fiber.

Devices of the invention also include a flow sensor for measuring the blood velocity. Any type of flow sensor may be used with the invention, although in certain embodiments, non-optical sensor are used to measure flow. For example, the flow sensor may include an electronic ultrasonic transducer that contains piezoelectric elements for determining flow. In certain aspects, the flow sensor can determine velocity by taking Doppler velocity measurements.

The provided devices can be used in variety of settings where assessment of vessel pressure and/or flow is needed. Devices of the invention are particularly useful for determining blood pressure and velocity in arteries, especially coronary arteries. The provided devices can also be used for pressure-volume loop analysis for hemodynamic assessment. In addition, devices of the invention can also be used as a guidewire for therapy-delivering catheters. In certain aspects of the invention, a method for determining pressure and/or flow is provided. The method involves inserting a device into a vessel and determining pressure and/or flow with the device. The device involves an elongated body, preferably a guidewire, with pressure and flow sensors positioned thereon. The pressure sensor of the device incorporates optical fibers, rather than resistive elements, thus providing greater accuracy and resistance to drift.

### BRIEF DESCRIPTION OF THE DRAWINGS

FIG. 1 depicts an optical fiber suitable for use with the provided imaging devices.

FIG. 2 depicts a pressure transducer based on a Fabry-Perot interferometer.

FIG. 3 depicts an embodiment of sensor that includes a piezoelectric element.

FIG. 4 depicts an imaging element that uses Fiber Bragg Gratings.

FIG. 5 depicts an imaging element that uses Fiber Bragg Gratings.

FIG. 6 illustrates an exemplary optical fiber configuration.

FIG. 7 illustrates a second exemplary optical fiber configuration.

FIG. 8 illustrates an exemplary guidewire configuration.

FIG. 9 illustrates an exemplary guidewire configuration for use in practicing the invention from a close-up, cross-sectional perspective.

### DETAILED DESCRIPTION

The invention generally relates to devices for determining pressure and flow in a vessel and methods of using such devices. The device can include an elongate body configured for insertion into a vessel, a flow sensor positioned on the elongate body configured for detecting flow in the interior of the vessel, and a pressure sensor positioned on the elongate body configured for detecting pressure in the interior of the vessel. Methods of using such devices can involve inserting the aforementioned device into a vessel, such as a blood vessel, and measuring pressure and/or flow with the device. The devices provided herein provide greater benefit to a physician by combining multiple features into one product. Moreover, the provided devices are less sensitive to pressure drift when compared to conventional devices.

Although devices of the present invention are suitable for use with any elongated body, in certain embodiments, the invention encompasses an imaging catheter or guidewire. The provided catheter or guidewire is configured for intraluminal introduction into a target body lumen. The dimensions and other physical characteristics of the catheter or guidewire may vary depending on the body lumen that is to be accessed. In addition, the dimensions can depend on the placement and number of imaging elements included on the imaging catheter or guidewire.

The provided catheters and guidewires may also serve other functions in addition to pressure and flow determination. In certain aspects, the provided catheter may also serve as a delivery catheter for delivery of some type of a therapeutic device, such as a stent, ablator, or balloon. During the procedure, the provided catheter may be used to identify the appropriate location and the delivery function used to deliver the device to the appropriate location. In certain embodiments, the provided guidewire may serve as rail for the introduction of a catheter. The catheter is slid over the provided guidewire and used as normal.

For embodiments encompassing a guidewire, the sensors can be formed as or be integrated into the body of the guidewire, circumscribe the guidewire, and/or run along the body of the guidewire. The provided guidewire may also include an outer support structure or coating surrounding the sensors. In certain embodiments, the sensors comprise an optical fiber. For example, the pressure sensor may be fiber optic based, as described below. In certain embodiment, the provided guidewire including the sensors (for example, an optical fiber and transducer material) and surrounding support structure can have a total outside diameter of less than 1 mm, preferably less than 300 micron (less than about 1 French).

The provided guidewire bodies may include a solid metal or polymer core. Suitable polymers include polyvinylchloride, polyurethanes, polyesters, polytetrafluoroethylenes (PTFE), silicone rubbers, natural rubbers, and the like. Preferably, at least a portion of the metal or polymer core and other elements that form the imaging guidewire body are flexible.

In certain embodiments, a catheter for detecting both pressure and flow is provided. In certain embodiments, the sensor, such as the pressure sensor, may comprise an optical fiber. The sensors can form or be integrated within the body of the catheter, circumscribe the catheter, placed on a distal end face of the catheter, and/or run along the body of the catheter. The provided catheter may also include an outer support structure or coating surrounding the detection elements. As encompassed by the invention, catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is preferably below 8 French, more preferably below 7 French, and most preferably in the range from 2 French to 7 French.

Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques. Preferably, at least a portion of the catheter body is flexible.

In certain embodiments, the provided guidewire of the invention includes a pressure sensor and a flow sensor. In other words, the guidewire is a combination guidewire that includes both pressure and flow sensing functions. Pressure sensors can be used to measure pressure within the lumen and flow sensors can be used to measure the velocity of blood flow. A guidewire with both a pressure sensor and a flow sensor provides a desirable environment in which to calculate fractional flow reserve (FFR) using pressure readings, and coronary flow reserve (CFR) using flow readings.

The ability to measure and compare both the pressure and velocity flow to determine an index of hyperemic steno sis resistance significantly improves the diagnostic accuracy of ischemic testing. It has been shown that distal pressure and velocity measurements, particularly regarding the pressure drop-velocity relationship such as Fractional Flow reserve (FFR), Coronary flow reserve (CFR) and combined P-V curves, reveal information about the stenosis severity. For example, in use, the guidewire may be advanced to a location on the distal side of the stenosis. The pressure and flow velocity may then be measured at a first flow state. Then, the flow rate may be significantly increased, for example by the use of drugs such as adenosine, and the pressure and flow measured in this second, hyperemic, flow state. The pressure and flow relationships at these two flow states are then compared to assess the severity of the stenosis and provide improved guidance for any coronary interventions. The ability to take the pressure and flow measurements at the same location and same time with the combination tip sensor, improves the accuracy of these pressure-velocity loops and therefore improves the accuracy of the diagnostic information.

A pressure sensor allows one to obtain pressure measurements within a body lumen. A particular benefit of pressure sensors is that pressure sensors allow one to measure of FFR in vessel. FFR is a comparison of the pressure within a vessel at positions prior to the stenosis and after the stenosis. The level of FFR determines the significance of the stenosis, which allows physicians to more accurately identify clinically relevant stenosis. For example, an FFR measurement above 0.80 indicates normal coronary blood flow and a non-significant stenosis. Another benefit is that a physician can measure the pressure before and after an intraluminal intervention procedure to determine the impact of the procedure.

A pressure sensor can be mounted on the distal portion of a flexible elongate member. In certain embodiments, the pressure sensor can be formed of a crystal semiconductor material having a recess therein and forming a diaphragm bordered by a rim. A reinforcing member is bonded to the crystal and reinforces the rim of the crystal and has a cavity therein underlying the diaphragm and exposed to the diaphragm. A resistor having opposite ends is carried by the crystal and has a portion thereof overlying a portion of the diaphragm. Electrical conductor wires can be connected to opposite ends of the resistor and extend within the flexible elongate member to the proximal portion of the flexible elongate member. Additional details of suitable pressure sensors that may be used with devices of the invention are described in U.S. Pat. No. 6,106,476, which also describes methods for mounting the pressure sensor 104 within a sensor housing. As discussed in further detail below, however, the pressure sensor can also be fiber optic-based.

In certain aspects, the guidewire of the invention also includes a flow sensor. The flow sensor can be used to measure blood flow velocity within the vessel, which can be used to assess coronary flow reserve (CFR). The flow sensor can be, for example, an ultrasound transducer, a Doppler flow sensor or any other suitable flow sensor, disposed at or in close proximity to the distal tip of the guidewire. The ultrasound transducer may be any suitable transducer, and may be mounted in the distal end using any conventional method, including the manner described in U.S. Pat. Nos. 5,125,137, 6,551,250 and 5,873,835. In certain aspects, the ultrasonic transducer is an electronic, rather than a fiber optic sensor. It is also contemplated, however, that the flow sensor can be fiber-optic based as well.

The provided guidewire may also be connected to an instrument, such as a computing device (e.g. a laptop, desktop, or tablet computer) or a physiology monitor, which converts the signals received by the sensors into pressure and velocity readings. The instrument can further calculate Coronary Flow Reserve (CFR) and Fractional Flow Reserve (FFR) and provide the readings and calculations to a user via a user interface.

As noted above, the pressure sensor may comprise a fiber optical sensor. An optical fiber consists of a thin, low-loss glass wire with a center or core region having a slightly higher refractive index than its surrounding region or cladding. Light is guided inside the core region by total internal reflection at the core-cladding interface. Depending in the size of the core region, one single or multiple light paths (modes) are permitted to propagate, referred to as single-mode or multimode fiber. Typically, the bare optical fiber has an outer diameter of 125 um with a core diameter of 9 um in the case of single-mode fibers and 50 um or 62.5 um for multimode fibers. Different protective coatings may be applied to protect the fiber from mechanical damage.

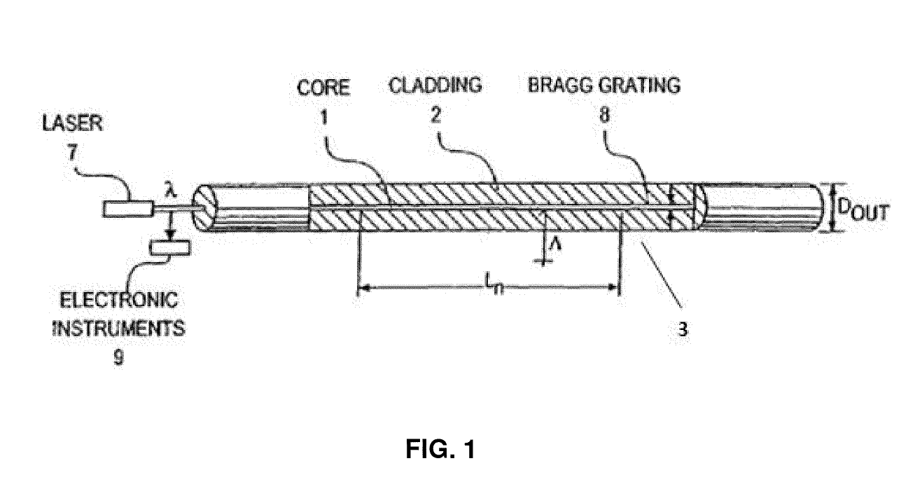

An exemplary optical fiber for use with the invention is provided in FIG. 1. The optical fiber 3 may be a single mode optical fiber. The optical fiber 3 includes a core 1, a cladding 2, and a Fiber Bragg Grating 8. The optical fiber 3 is coupled includes a laser 7. The Bragg Grating 8 will reflect back a narrowband component centered about the Bragg wavelength λ given by λ=2nΛ, where n is the index of the core of the fiber and A represents the grating period. With a tunable laser 7 and different grating periods (each period at approximately 0.50μ) at different positions on the fiber, it is possible to make independent measurements in each of the grating positions. As used in the provided catheters and guidewires, the optical fiber 3 with Fiber Bragg Grating 8 helps transmit light within the optical fiber for the determination of pressure.

Fiber optic-based sensors offer numerous benefits over conventional electronic sensors including their small size, which makes them ideal for embedding and surface mounting, and their high degree of biocompatibility. In addition, their non-intrusive nature and electromagnetic immunity makes them ideal for cardiovascular medical applications. Any type of fiber optical sensor can be used with the present invention. Specific types of compatible fiber optical sensors include point sensors, in which the measurement is carried out at a single point in space, but possible using multiple channels for addressing multiple points. Exemplary point sensors include Fabry-Perot sensors and single Fiber Bragg Grating (FBG) sensors. Suitable fiber optical sensors include integrated sensors, in which the measurement averages a physical parameter over a certain spatial section and provides a single value. Exemplary integrated sensors include a deformation sensor which measures strain over a long base length. Additional types of fiber optical sensors include quasi-distributed or multiplexed sensors. In these types of sensors, the measurement is determined at a number of fixed, discrete points along a single fiber optical cable. The most common example are multiplexed FBGs. Further fiber optical sensors include distributed sensors, in which the parameter of interest is measured along a single optical cable. Examples of such sensors include systems based on Rayleigh, Raman, and Brillion scattering.

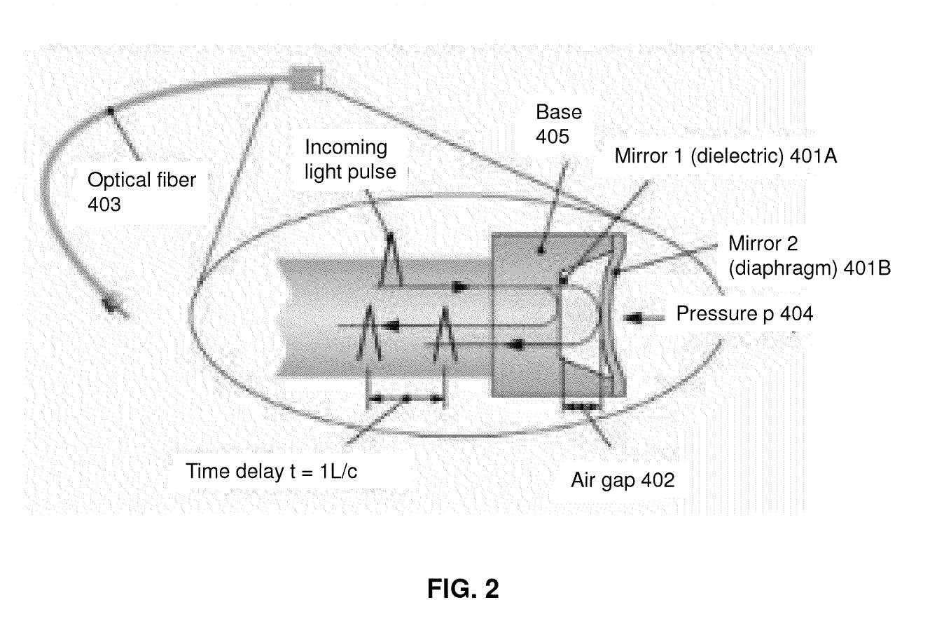

In certain aspects of the invention, the optical fiber based pressure sensor includes a Fabry-Perot cavity. As shown in FIG. 2, the pressure sensor Fabry-Perot cavity can comprise of a pair of parallel mirrors 401A and 401B separated by an air gap Ls 402. This arrangement is referred to as a Fabry-Perot cavity or sensing interferometer. A first semi-reflective mirror 401A is formed by depositing a dielectric layer at the end of the optical fiber 403. A second mirror 401B is formed by a diaphragm mounted in front of the optical fiber 403. Exposing the diaphragm to the pressure p to be measured changes the gap Ls. Hence, by measuring Ls 402, the applied pressure can be determined. Different pressure ranges can be accommodated by appropriately selecting thickness and diameter of the diaphragm to keep the maximum deflection of similar value and maintain a linear relation between pressure and deflection.

In certain embodiments, the optical fiber based pressure sensor includes a Fiber Bragg Grating (FBG) within the optical fiber. Fiber Bragg Gratings (FBGs) provide a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interference in the beam. If the paths are identical, then the two paths combine to form the original beam. If the paths are different, then the two parts will add or subtract from each other and form an interference. The Fiber Bragg Grating elements are thus able to sense a change in the wavelength between the constant path and the change path based on received ultrasound or acoustic energy. As contemplated by the invention, a change in pressure may also induce a readable change between the constant path and the change path based on received ultrasound or acoustic energy.

In certain embodiments, the pressure sensor includes a piezoelectric element to generate the acoustic or ultrasound energy. In such aspect, the optical fiber of the pressure sensor may by coated by the piezoelectric element. The piezoelectric element may include any suitable piezoelectric or piezo-ceramic material. In one embodiment, the piezoelectric element is a poled polyvinylidene fluoride or polyvinylidene di-fluoride material. The piezoelectric element can be connected to one or more electrodes that are connected to a generator that transmits pulses of electricity to the electrodes. The electric pulses cause mechanical oscillations in the piezoelectric element, which generates an acoustic signal. Thus, the piezoelectric element is an electric-to-acoustic transducer. Primary and reflected pulses (i.e. reflected from the imaging medium) are received by the Bragg Grating element and transmitted to an electronic instrument to determine pressure in a vessel.

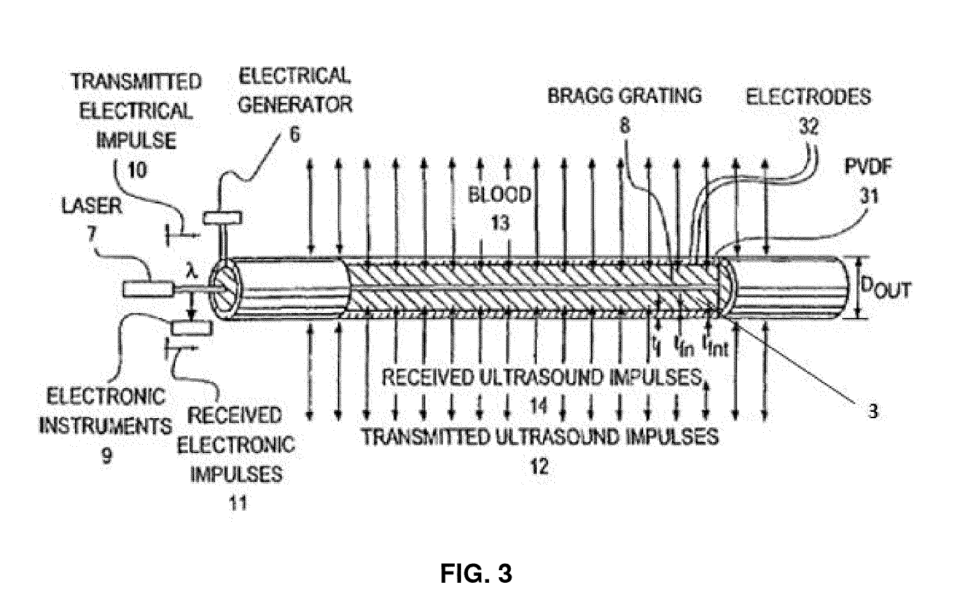

FIG. 3 depicts an embodiment of a fiber-optic based pressure sensor that comprises a piezoelectric element. The pressure sensor includes an optical fiber 3 (such as the optical fiber in FIG. 1) with Fiber Bragg Grating 8 and a piezoelectric element 31. As shown in FIG. 3, an electrical generator 6 stimulates the piezoelectric element 31 (electrical-to-acoustic transducer) to transmit ultrasound impulses 10 to both the Fiber Bragg Grating 8 and the outer medium 13 in which the device is located. For example, the outer medium may include blood when determining pressure within a vessel. Primary and reflected impulses 11 are received by the Fiber Bragg Grating 8 (acting as an acoustic-to-optical transducer). The mechanical impulses deform the Bragg Grating and cause the Fiber Bragg Grating to modulate the light reflected within the optical fiber, which generates an interference signal. The interference signal is recorded by electronic detection instrument 9, using conventional methods. The electronic instrument may include a photodetector and an oscilloscope. Imaging information regarding the contact between the imaging device and the object can be generated from these recorded signals. The electronic instruments 9 modulation of light reflected backwards from the optical fiber due to mechanical deformations. The optical fiber with a Bragg Grating described herein and shown in FIG. 1, the imaging element described herein and shown in FIG. 3 and other varying embodiments are described in more detail in U.S. Patent Nos. 6,659,957 and 7,527,594 and in U.S. Patent Publication No. 2008/0119739.

In another aspect, the pressure sensor does not require an electrical-to-acoustic transducer to generate acoustic/ultrasound signals. Instead, the pressure sensor utilizes the one or more Fiber Bragg Grating elements of the optical fiber in combination with an optical-to-acoustic transducer material to generate acoustic energy from optical energy. The acoustic-to-optical transducer (signal receiver) may also act as an optical-to-acoustic transducer (signal generator).

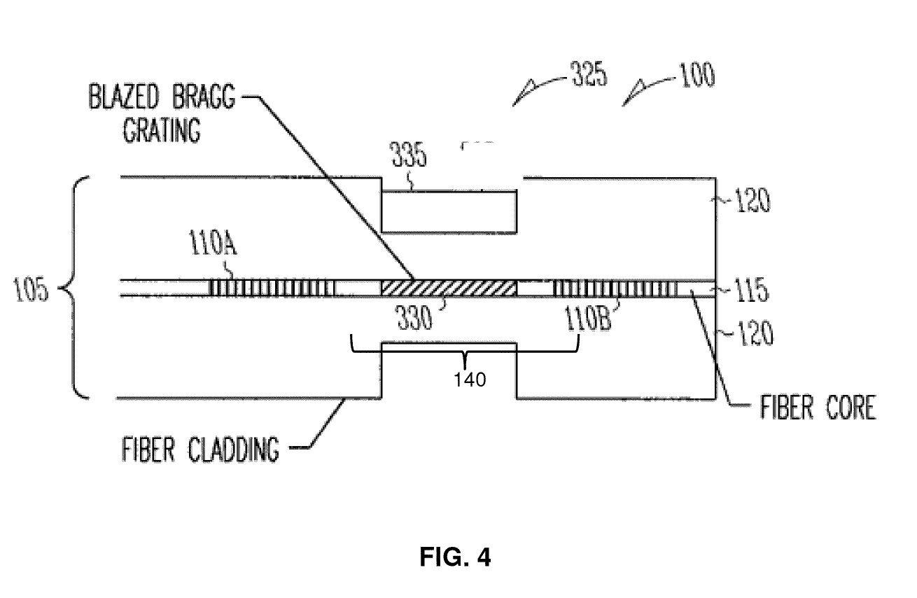

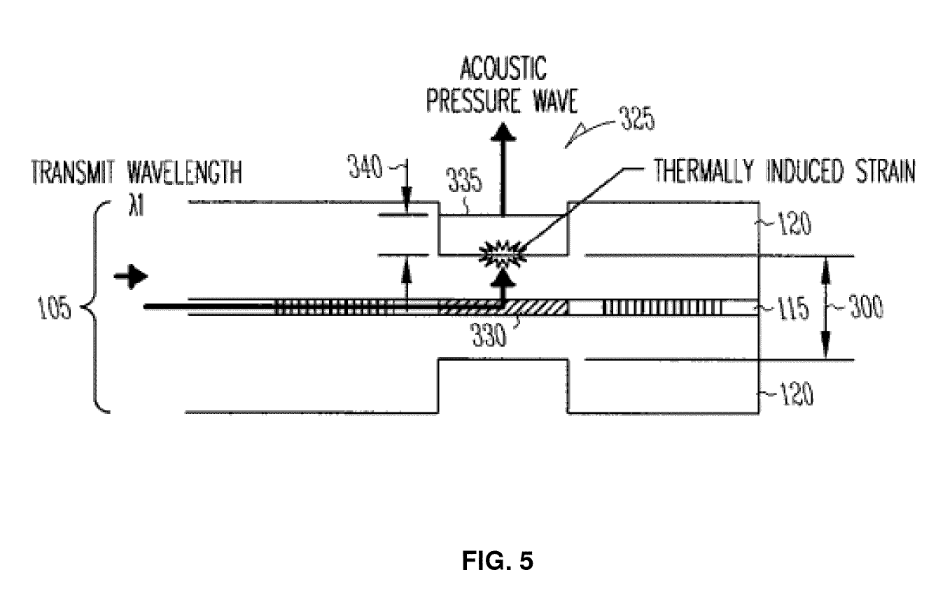

To generate the acoustic energy, pressure sensor may include a combination of blazed and un-blazed Fiber Bragg Gratings. Un-blazed Bragg Gratings typically include impressed index changes that are substantially perpendicular to the longitudinal axis of the fiber core of the optical fiber. Un-blazed Bragg Gratings reflect optical energy of a specific wavelength along the longitudinal of the optical fiber. Blazed Bragg Gratings typically include obliquely impressed index changes that are at a non-perpendicular angle to the longitudinal axis of the optical fiber. Blazed Bragg Gratings reflect optical energy away from the longitudinal axis of the optical fiber. FIGS. 4 and 5 depict sensors according to this embodiment.

FIG. 4 shows an example of a sensor that uses Fiber Bragg Gratings to generate acoustic energy. As depicted in FIG. 4, the sensor 100 includes an optical fiber 105 with un-blazed Fiber Bragg Grating 110A and 110B and blazed Fiber Bragg Grating 330 and a photoacoustic material 335 (optical-to-acoustic transducer). The region between the un-blazed Fiber Bragg Grating 110A and 110B is known as the strain sensing region 140. The strain sensing region may be, for example, 1 mm in length. The Blazed Fiber Bragg Grating 330 is implemented in the strain sensing region 140. The photoacoustic material 335 is positioned to receive the reflected optical energy from the blazed Fiber Bragg Grating 330. Although not shown, the proximal end of the optical fiber 105 is operably coupled to a laser and one or more electronic detection elements.

In operation and as depicted in FIG. 4, the blazed Fiber Bragg Grating 330 receives optical energy of a specific wavelength λ1 from a light source, e.g. a laser, and blazed Grating 330 directs that optical energy towards photoacoustic material 335. The received optical energy in the photoacoustic material 335 is converted into heat, which causes the material 335 to expand. Pulses of optical energy sent to the photoacoustic material 335 cause the photoacoustic material 335 to oscillate. The photoacoustic material 335 oscillates, due to the received optical energy, at a pace sufficient to generate an acoustic or ultrasound wave. The acoustic wave is transmitted out to and reflected from the object surface back to the sensor, particularly in response to the pressure in the vessel. The acoustic wave reflected from the object surface impinges on photoacoustic transducer 335, which causes a vibration or deformation of photoacoustic transducer 335. This results in a change in length of light path within the strain sensing region 140.

Light received by blazed fiber Bragg grating from photoacoustic transducer 335 and into fiber core 115 combines with light that is reflected by either fiber Bragg grating 110A or 110B (either or both may be including in various embodiments). The light from photoacoustic transducer 135 will interfere with light reflected by either fiber Bragg grating 110A or 110B and the light returning to the control unit will exhibit an interference pattern. This interference pattern encodes the pressure differential captured by pressure sensor 100. The light can be received into photodiodes within a control unit and the interference pattern thus converted into an analog electric signal. This signal can then be digitized using known digital acquisition technologies and processed, stored, or displayed as a readout of the target treatment site.

Acoustic energy of a specific frequency may be generated by optically irradiating the photoacoustic material 335 at a pulse rate equal to the desired acoustic frequency. The photoacoustic material 335 can be any suitable material for converting optical energy to acoustic energy and any suitable thickness to achieve a desired frequency. The photoacoustic material 335 may have a coating or be of a material that receives acoustic energy over a band of frequencies to improve the generation of acoustic energy by the photoacoustic material and reception of the acoustic energy by the optical fiber imaging region.

In one example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In a further example, the photoacoustic material is of a thickness 300 that is about ¼ the acoustic wavelength of the material at the desired acoustic transmission/reception frequency, and the corresponding glass-based optical fiber imaging region resonant thickness 300 is about ½ the acoustic wavelength of that material at the desired acoustic transmission/reception frequency. This may improve the generation of acoustic energy by the photoacoustic material and reception of the acoustic energy by the optical fiber imaging region. A suitable photoacoustic material is pigmented polydimethylsiloxane (PDMS), or a mixture of PDMS, carbon black, and toluene.

The photoacoustic sensor described and depicted in FIGS. 3 and 4 and other varying embodiments are described in more detail in U.S. Pat. Nos. 7,245.789, 7447,388, 7,660,492, 8,059,923 and in U.S. Patent Publication Nos. 2010/0087732 and 2012/0108943.

In certain embodiments, an optical fiber of a imaging element (e.g., see FIGS. 3-5) can include a plurality of Fiber Bragg Gratings, each with its own unique period (e.g., 0.50μ), that interact with at least one other transducer. Because each Fiber Bragg Grating can be directed to transmit and receive signals of specific wavelengths, the plurality of Fiber Bragg Gratings in combination with a tunable filter can be used to generate an array of distributed sonars.

In certain aspects, one or more optical pressure sensors are incorporated into the provided guidewire. The provided guidewire allows one to detect both pressure and flow in a vessel prior to introducing a catheter into a body lumen, e.g., a blood vessel. The one or more optical pressure sensors can be formed around an inner guidewire body, integrated into an inner guidewire body, or form the guidewire body itself. The provided guidewire may include a support structure covering at least a portion of the pressure sensors. The support structure can include one or more windows that allow the pressure sensor to send and receive signals that form the pressure data. In certain embodiments, a plurality of pressure sensors surrounds an inner guidewire body. In this configuration, the pressure sensors are placed next to each other, parallel to, and along the length of the inner guidewire body. The optical fibers comprising the pressure sensors can be optionally overlaid with a protective outer coating that provides for transmission of imaging signals.

Typically, the optical fibers comprising the pressure sensors are placed parallel to and along the length of the guidewire. In certain aspects, the pressure sensors detect pressure substantially perpendicular to the longitudinal axis of the provided guidewire. However, other configurations may be used. For example, one or more pressure sensors may be wrapped around the inner guidewire body. In addition, it is also contemplated at least a portion of the pressure sensors are positioned substantially across the longitudinal axis of the guidewire. For example, the pressure sensor can be positioned across a distal tip of the provided guidewire such that the pressure sensor detects pressure in front of the imaging guidewire.

The guidewire of the invention may be used in conjunction with any type of catheters, including delivery catheters. Furthermore, the provided catheters are suitable for use with any type of guidewire. The provided catheter allows an operator to obtain both pressure and flow measurements in a vessel as the catheter is slidably moved along a guidewire to the location of interest. In certain embodiments, the provided catheter is also a delivery catheter that can perform intraluminal procedures such as delivering implants, ablation, and extraction Like the provided guidewire, the provided catheter includes one or more imaging elements. As discussed previously, each pressure sensor may include an optical fiber that may comprise a Fiber Bragg Grating Like the provided guidewire, the pressure sensors can be positioned anywhere along and on the inner body of the provided catheter.

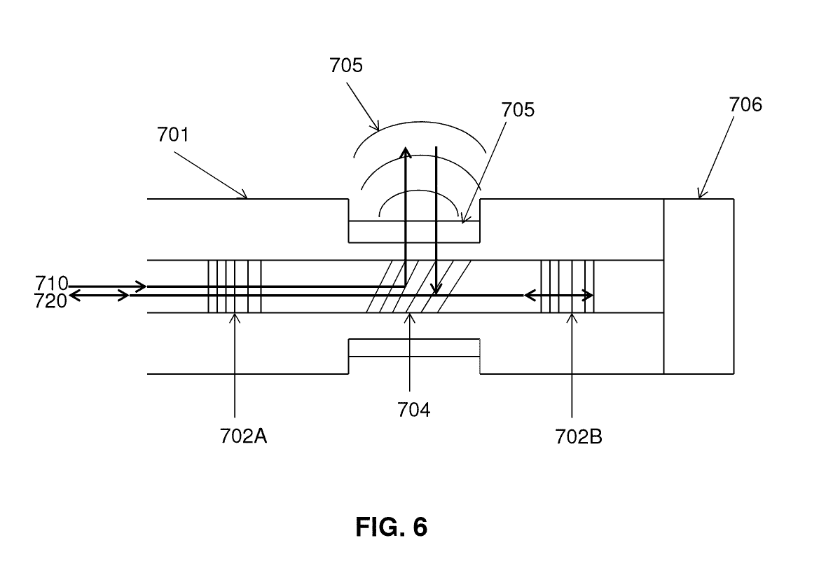

Reference will now be made to FIGS. 6-9, which depict certain exemplary embodiments of the invention. While the embodiments illustrated in these figures pertain primarily to guidewires, it is to be understood that the concepts demonstrated are equally applicable to other elongated bodies, such as catheters.

FIG. 6 depicts an exemplary configuration for flow and pressure measurement in a single device. The exemplary optical fiber of the configuration can be easily incorporated into a guidewire or catheter, as explained in further detail below. As shown, a single-mode optical fiber 701 is configured with multiple sensors for measurement of blood flow velocity and measurement of blood pressure. Etched into the optical fiber 701 are two un-blazed FBGs 702a and 702b. Between the two un-blazed FBGs 702a and 702b is a blazed FBG 704 etched into the optical fiber 701. In this embodiment, the blazed FBG 704 is used for measurement of pressure. On either side of the blazed FBG 704 is a layer of optically absorptive photoacoustic material 705 for converting light into sound waves and vice versa. At the distal end of the optical fiber 701 is a flow sensor 706, which in certain embodiments, comprises an electronic ultrasonic transducer. Such ultrasonic transducers and their use in measuring flow are known in the art, for example, the Medical Ultrasonic Sensor for Blood Flow Meter (Model PW-2) from CNIRHurricane Tech (Shenzhen) Co.

Although the provided optical fiber configuration can be of any size and dimension, in certain embodiments, the outer dimensions of the optical fiber and flow sensor are such that the optical fiber can be incorporated into a hollow 0.014″ interventional guidewire.

Reference will now be made to the operation of the device. For pressure measurement, Power light 710 is supplied as pulses which match the desired frequency of ultrasound. The Power light 710 is diffracted by the blazed FBG 704 toward the optically absorptive photo acoustic material 705, which converts the light pulses into ultrasound 740. Regular FBGs transmit or reflect a percentage of or all light of certain wavelengths. Blazed FBGs, on the other hand, diffract a percentage of or all light of certain wavelengths.

Ultrasound 740 is emitted from the photoacoustic material 705 and is reflected back by blood cells circulating in the vessel. This reflected sound is converted to signal light 720 by the photoacoustic material 705. The signal light 720 travels down the optical fiber 701 to a first un-blazed FBG 702a, which reflects a portion of the signal light 720 back to the source, and second un-blazed FBG 702b, which reflects the remaining signal light 720 back to the source. The ultrasound reflection from the surrounding environment the properties of the signal light passing between the two un-blazed FBGs 702a and 702b, which acts as an interferometer. Externally, the modified signal light is compared to the unmodified signal light in between ultrasound pulses and the pressure data associated with the circulating blood cells is extracted. Further detail on the implementation of blazed FBGs in optical sensors is provided in U.S. Pat. No. 7,245,789, incorporated herein by reference.

The flow sensor also works by sending out ultrasound waves, which when reflected by moving blood cells, may be analyzed by a Doppler blood flow analyzer, for example. As the signal returns, Doppler flow signal processing is performed on the signal to measure the phase shift of the reflected ultrasound. In contrast to the pressure sensor, in which signals are transmitted via the optical fiber, the signals from the flow sensor are transmitted via an electrical connection wire, in certain embodiments. Exemplary configurations for the electrical connection wires are provided in further detail below. In certain embodiments, however, the flow sensor is also an optical sensor and can actually use the same components as the pressure sensor. Although the different functions can use the same optical sensor described above, the signal processing will differ according to use, such as in multiplexed operation. The nature of the emitted sound wave may also be adjusted depending on the intended use.

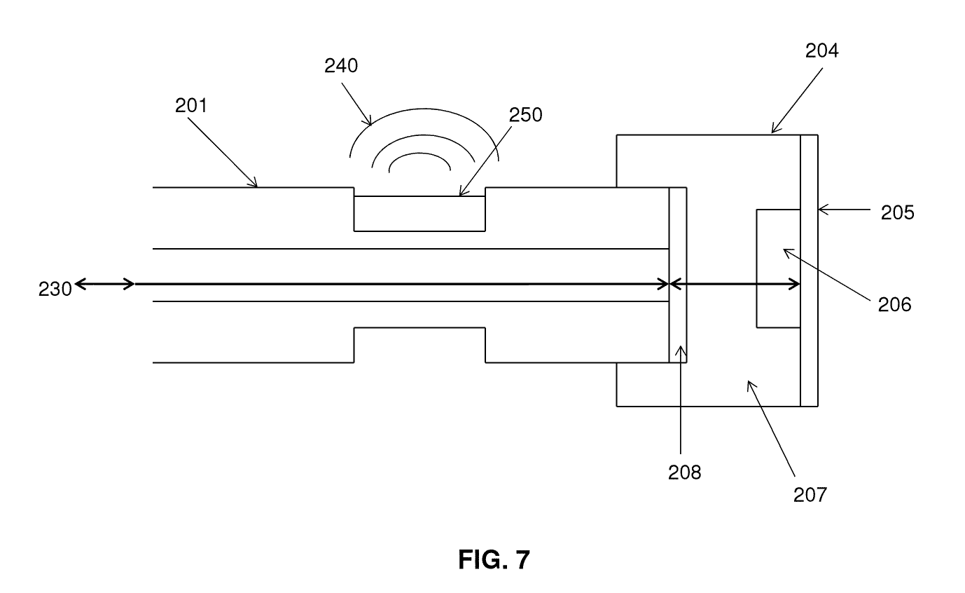

FIG. 7 depicts yet another exemplary optical fiber configuration for imaging as well as flow and pressure measurement. Again, the exemplary optical fiber can be easily incorporated into a guidewire or catheter, as explained in further detail below. As shown, a single-mode optical fiber 201 is configured with multiple sensors for measurement of blood flow velocity and measurement of blood pressure.

Once again, the optical fiber facilitates the measurement of pressure, although through different means as the previous embodiment. At the distal end of the optical fiber 201 is a pressure sensor housing 204. The pressure sensor housing 204 includes a diaphragm 205 and a vacuum chamber 206 located behind the diaphragm 205. The pressure sensor 204 also comprises a MEMS structure 207 and a partial mirror 208. Pressure sensors suitable for use with the invention are known in the art. One such pressure sensor, the OPP-M25, is manufactured Opsens, Inc. For measurement of flow, the optical fiber features a flow sensor 250, which in certain embodiments, comprises an electronic ultrasonic transducer. In this configuration, the flow sensor is positioned in a recess of the cladding of the optical fiber 201.

Although the provided optical fiber configuration can be of any size and dimension, in certain embodiments, the optical fiber 201 has an outer diameter of approximately 0.006″ and the pressure sensor 204 has an outer diameter of up to 0.0010,″ allowing the incorporation of the optical fiber 201 into a hollow 0.014″ interventional guidewire.

Reference will now be made to the operation of the device. In this embodiment, pressure measurement is again based on a Fabry-Perot interferometer. To detect pressure, light from the source (Pressure light 230) travels down the length of the optical fiber 201 and is partially reflected by the partial mirror 208. The remaining pressure light 230 continues through the MEMS structure 207 and vacuum chamber 206 and is reflected back to the source by the diaphragm 250. The pressure sensor 204 is calibrated such that a known change in the length of the light path corresponds to a specific external pressure. The light path through the vacuum chamber 206 changes as the external pressure is applied to the diaphragm 205, shortening the optical path.

The flow sensor 250 also works by sending out ultrasound waves 240, which when reflected by circulating blood cells, may be analyzed by a Doppler blood flow analyzer, for example. As the signal returns, Doppler flow signal processing is performed on the signal to measure the phase shift of the reflected ultrasound. In contrast to the pressure sensor 204, in which signals are transmitted via the optical fiber 201, the signals from the flow sensor 250 are transmitted via an electrical connection wire. Exemplary configurations for the electrical connection wires are provided in further detail below. In certain embodiments, however, the flow sensor is also an optical sensor and can actually use the same components as the pressure sensor. Although the different functions can use the same optical sensor described above, the signal processing will differ according to use. The nature of the emitted sound wave may also be adjusted depending on the intended use.

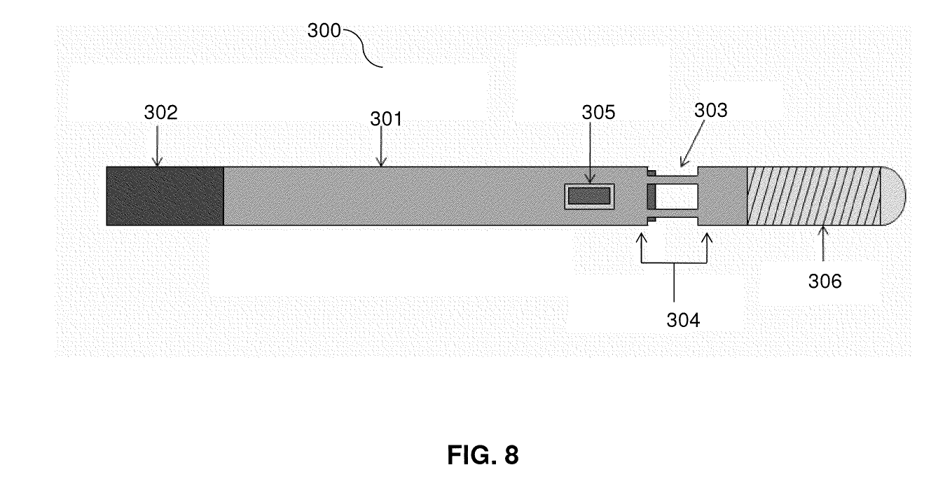

FIG. 8 shows a guidewire configuration for use with either fiber optic configuration described above. Guidewire 300 is hollow and composed primarily of hypotubes. Guidewire 300 may include a distal nitinol hypotube 301 joined (e.g., welded, by adhesives, other) to a proximal stainless steel hypotube 302. The distal end of the nitinol hypotube 301 has a cage structure 304 comprising windows 303 that allow the pressure or flow sensor to be exposed to the surrounding environment. The windows 303 can be manufactured using typical stent cutting techniques. In certain embodiments, the nitinol hypotube 301 may be slotted or cut in some other manner to improve flexibility. The nitinol hypotube 301 has one or more windows 305 at the distal end but proximal to the cage 304, that serve as openings for pressure/flow ultrasound. In certain embodiments, these windows 305 might be covered or filled with a material that is transparent to ultrasound. As contemplated by the invention, there are two window regions 303 and 305 to accommodate the two sensors. In some embodiments, a short radiopaque tip coil 306 is attached to the distal end of the nitinol hypotube 301 via a distal core that also contributes flexibility and shape-ability to the distal end of the guidewire 300. In certain aspects, any length of the guidewire may be coated with a hydrophilic or other lubricious coating. At the proximal end of the guidewire 300, the optical fiber installed inside could terminate with a flush orientation suitable for connecting to an optical receiver or could have a variety of methods for transmitting the light signal out of the radial surface of the hypotube to an appropriate receiver.

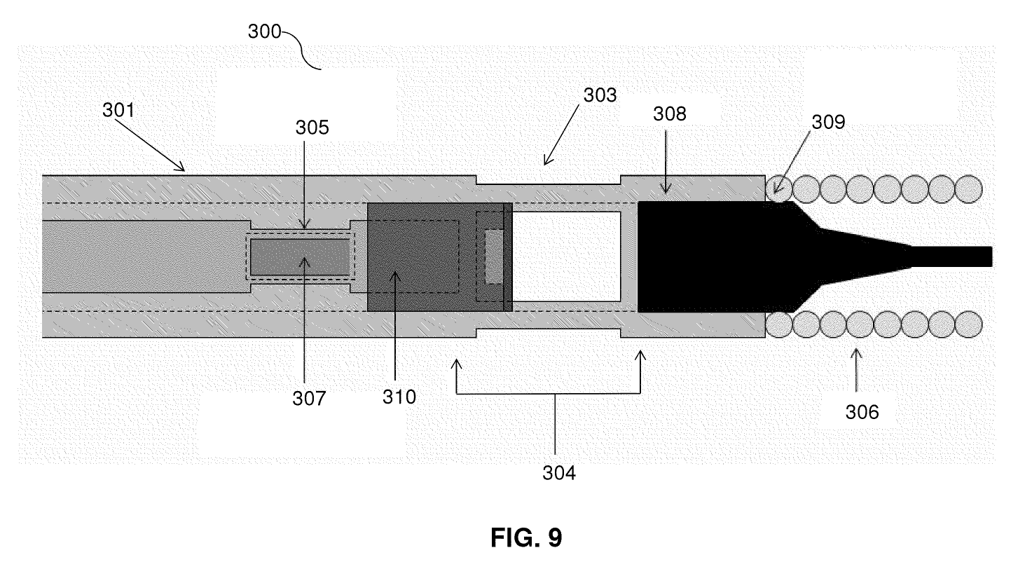

The above device can be prepared in a variety of ways. For example, a nitinol hypotube may be pre-cut with the aforementioned cage and windows, and joined to a stainless steel hypotube through convention welding. An optical fiber can be inserted through the distal end of the nitinol hypotube and aligned with the pressure sensor in the cage and the electronic ultrasound transducer 310 located at the flow window. The flow window 305 may expose the electronic ultrasound transducer 307, as shown in FIG. 9. Once inserted, the optical fiber can be secured using an adhesive. Next, the tip coil can be soldered to the distal core 308 as a subassembly, as shown in FIG. 9. The tip coil subassembly is then glued onto the distal end of the nitinol hypotube 301. A shoulder 309 on the core may be used to help attachment of the tip coil 306. At this stage, the entire guidewire or any portion thereof can be hydrophilically coated. It is to be noted that catheters comprising similar features may be prepared in a similar manner.

In certain embodiments, one or more electrical connection wires are couples to one or more sensors, such as the electronic flow sensor. The electrical connection wires can include a conductive core made from a conductive material, such as copper, and an insulative coating, such as a polyimide, fluoropolymer, or other insulative material. The electrical connection wires extend from one or more sensors located on the distal end of the guidewire, run down the length of the guidewire, and connect to a connector housing at a proximal end.

Any suitable arrangement of the electrical connection wires through the length of the elongate member can be used. The arrangement of electrical connection wires must provide for a stable connection from the proximal end of the guidewire to the distal end of the guidewires. In addition, the electrical connection wires must be flexible and/or have enough slack to bend and/or move as the guidewire is moved.

In certain embodiments, the proximal end of the electrical connection wires connects to a connector housing. For example, the electrical connection wires are joined together to form a male connector at a proximal end. The male connector mates with a female connector of the connector housing. The connector housing may be connected to an instrument, such as a computing device (e.g., a laptop, desktop, or tablet computer) or a physiology monitor, which converts the signals received by the sensors into pressure and velocity readings. The instrument can further calculate Coronary Flow Reserve (CFR) and Fractional Flow Reserve (FFR) and provide the readings and calculations to user via a user interface.

The invention also encompasses methods of using the provided device to determine pressure and/or flow inside a vessel. The method may involve introducing a device into a vessel and measuring pressure and/or flow inside the vessel with the device. The contemplated devices have already been described at length above, however, in certain embodiments, the device includes an elongate body configured for insertion into a vessel and a flow sensor and pressure sensor positioned on the device. As encompassed by the invention, the pressure sensor comprises an optical sensor.

In practice, the method may also involve injecting a local anesthetic into the skin to numb the area of the patient prior to surgery. A puncture is then made with a needle in either the femoral artery in the groin or the radial artery in the wrist before the provided guidewire is inserted into the arterial puncture. Once positioned, the provided guidewire may then be used to measure pressure and/or flow in the vessel, techniques which are already well-known in the art. The provided methods also encompass the determination of FFR, CFR, and pressure-volume loop analysis for hemodynamic assessment.

After the guidewire has been positioned, a plastic sheath (with a stiffer plastic introducer inside it) is then threaded over the wire and pushed into the artery. The method may further involve inserting a catheter over the provided guidewire and advancing the catheter towards the heart. Once the catheter is in place, it can be used to perform a number of procedures including angioplasty, PCI (percutaneous coronary intervention) angiography, balloon septostomy, and an Electrophysiology study or ablation procedure.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A device for determining pressure and flow in a vessel, the device comprising:
an elongate body configured for insertion into a vessel;
a flow sensor positioned on the elongate body configured for detecting flow in the interior of the vessel; and
a pressure sensor positioned on the elongate body configured for detecting pressure in interior of the vessel, wherein the pressure sensor comprises an optical sensor.

2. The device of claim 1, wherein the vessel is a blood vessel.

3. The device of claim 1, wherein the elongate body is a catheter.

4. The device of claim 1, wherein the elongate body is a guidewire.

5. The device of claim 4, wherein the guidewire comprises a hollow guidewire.

6. The device of claim 1, wherein the pressure sensor comprises at least one optical fiber.

7. The device of claim 6, wherein the optical fiber is located inside the guidewire.

8. The device of claim 6, wherein the optical fiber comprises a Fabry-Perot cavity.

9. The device of claim 6, wherein the optical fiber comprises a fiber Bragg grating.

10. The device of claim 1, wherein the flow sensor comprises an ultrasonic transducer.

11. The device of claim 10, wherein the ultrasonic transducer is an electronic ultrasonic transducer.

12. The device of claim 1, wherein the flow sensor is positioned at a distal end of the elongate body.

13. The device of claim 1, wherein the pressure sensor is positioned at a distal end of the elongated body.

14. The device of claim 4, wherein the guidewire comprises a hydrophilic coating.

15. The device of claim 4, wherein the guidewire comprises a nitinol hypotube.

16. The device of claim 1, further comprising a tip coil positioned at the distal tip of the guidewire.

17. The device of claim 16, wherein the tip coil comprises a radiopaque tip coil.

18. A method for determining pressure or flow in a vessel, the method comprising:
introducing a device into a vessel, the device comprising
an elongate body configured for insertion into a vessel,
a flow sensor positioned on the elongate body configured for detecting flow in the interior of the vessel, and
a pressure sensor positioned on the elongate body configured for detecting pressure in the interior of the vessel, wherein the pressure sensor comprises an optical sensor; and
measuring pressure and/or flow inside the vessel.

19. The method of claim 18, wherein the pressure sensor comprises at least one optical fiber.

20. The method of claim 19, wherein the optical fiber comprises a fiber Bragg grating.
